(19) United States
(12) Patent Application Publication
(10) Pub. No.: US 2007/0020719 A1
(43) Pub. Date: Jan. 25, 2007

(54) Selective culture medium for the isolation and/or detection of species in the streptococcus genus

(76) Inventors: Anabel Durán Vila, Habana (CU); Claudio Rodriguez Martinez, Habana (CU)

Correspondence Address: LACKENBACH SIEGEL, LLP, LACKENBACH SIEGEL BUILDING, 1 CHASE ROAD, SCARSDALE, NY, 10583, US

(21) Appl. No.: 10/541,299

(22) PCT Filed: Jan. 12, 2004
(86) PCT No.: PCT/CU04/00001

§ 371 Date: Aug. 18, 2006

(30) Foreign Application Priority Data
Jan. 10, 2003 (CU) 2003-0008

(51) Int. Cl. C12Q 1/04

(52) U.S. Cl. 435/34

(57) ABSTRACT

The present invention is related with the Microbiology in particular with a selective culture medium for the isolation and detection of species of Streptococcus genus. The proposed medium allows the identification of species of Streptococcus by the appearance of three different tonalities in the organisms to detect and the appearance of fluorescent emissions, besides changes of the coloration of the medium that offers the identification with a high degree of sensibility and specificity.

The medium includes specific ratios of mixtures of nutritive bases, rich in compounds of protein origin (polypeptides, peptides, proteoses, and aminoacids) and vitamins, which facilitate an abundant growth of the target species. In addition, the inclusion of the blood enzymatic hydrolyzate, guarantees the presence in the medium of blood-derived substances or of its fractions that are necessary for the Streptococcus growth, without interfering the identification.

### TECHNICAL SECTOR

The present invention is related with Microbiology and specifically with the clinical and veterinary diagnosis, with the food quality control and with the environmental contamination studies where the methods for the isolation and detection of Streptococcus species are needed.

The streptococci are considered as a fastidious microorganism genus whose identification is extremely important in the clinical diagnosis. Within the classification carried out by Lancefield there are numerous pathogenic species to human, such as: Streptococcus pyogenes, Streptococcus agalactiae, Streptococcus anginosus, Enterococcus avium, Enterococcus faecium, Enterococcus faecalis, causal agents of septicemia, neonatal meningitis, endocarditis and urinary tract infections (Giuseppe, N. y Vito Mar N., 1989. Diccionario de Bacteriologia Humana, edición española Menarini). Although the streptococci aren't the microorganisms that more frequently cause urinary sepsis, it is necessary their detection, because specifically hemolytic streptococci are the most frequent cause of neonate sepsis, so its early detection allows to administer intraparto antibiotic prevention and to prevent the perinatal infection by this microorganism (Bosch, J.; Murillo, S.; Rico, M. y Salgado, M. 1998. Utilidad de un medio selectivo disco-caldo para la detección de estreptococo del grupo B en la vagina. Enferm. Infecc. Microbiol. Clin. Vol 16(2)). The early incidence of neonatal sepsis is of 1.48-4.08/1,000 alive borned children with a mortality of 8.7-10.5% (Montero, R.; Barbadillo, F.; Anso, S.; Marrero, M.; Carpintero I.; Sastre E. y Alonso B.: Sepsis neonatal por Streptococcus agalactiae. Qué hacer ?. An-Esp-Pediatr. 1998; 48 (3), p. 288-92; Juncosa, T.; Bosch, J.; Dopico, E.; Guardia, C.; Lite, J.; Sierra, M.; Barranco, M.; Matas, L.; Sanchez, F.; Sanfeliu, I. y Vinas, L. 1998. Infección neonatal por Streptococcus agalactiae. Estudio multicéntrico en el área de Barcelona. Enferm-Infecc-Microbiol-Clin.; 16(7), p. 312-5). The causal agent of this illness is mostly Streptococcus agalactiae, habitual resident of the female genital tract. Overmore new, not known urinary pathogens, as Streptococcus urinalis sp.nov., also appeares, that although have similarity with Streptococcus pyogenes and Streptococcus canis. It is considered a new species of Streptococcus genus and it's taxonomical denomination is Streptococcus urinalis sp.nov., strain type CCUG 41590T (Collins, M. D.; Hutson, R. A.; Falsen, E.; Nikolaitchouk, N.; La Claire, L. and Facklam, R. R. 2000. An unusual Streptococcus from human urine, Streptococcus urinalis sp. nov. Int J Syst Evol Microbiol; 50 Pt 3, p. 1173-1178).

Actually, there are in the market many culture media that are designed to isolate and/or to detect different species of the Streptococcus genus. Among them can be mentioned: Blood Agar, Blood Columbia Agar, Tryptone Soja Agar with Blood, Brain Heart Agar, and other (Manual of cultivation Means OXOID. 1995. UNIPATH, España; MERCK Microbiology Manual. 2000. Merck KgaA, Darmstadt; Manual DIFCO de Bacteriologia. 1984. Décima Edición, Francisco Soria Melguizo, S. A., Madrid).

These non-selective media do not allow the differentiation between pathogenic and no-pathogenic species. These media are not selective and allow the growth of other Gram-positive and Gram-negative bacteria that compete with the streptococci for the nutrients. In case of Proteus, for example, the strains that develop “spread” impede the isolation of the target colonies.

There are not highly nutrient substances in their formulations, thus it is necessary the blood addition which hinders the manipulation and increases the risk of contamination. In addition it doesn't allow to extend the expiration date of the medium, because the blood is biologically unstable compound.

The CLED medium, offered by the previously mentioned firms, is used widely in the laboratories and it is considered as one of the best options. It allows the growth of all urinary pathogens, Streptococcus among them, and other Gram-positive microorganisms that cannot be differentiated, because they possess the same cultural characteristics. Streptococcus growth is slow. The enterococci (Enterococcus faecalis, Enterococcus faecium and Enterococcus avium) can be confused with Shigella species.

In the MERCK Manual of culture media (MERCK Microbiology Manual, 2000. Merck KgaA, Darmstadt) are described an agar medium and a broth for the selective enrichment of Streptococcus that uses the sodium azide and the sodium sulfite as inhibitors of Gram-negative organisms. As disadvantages of theese media can be mentioned their high toxicity by the presence of the sodium azide (highly toxic ingredient) and the impossibility of differentiating between the species in the broth, which is used specifically to promote the microbial growth.

The OXOID manual (Manual of culture media, OXOID, 1995. UNIPATH, Spain) describes a selective medium for Streptococcus that requires the addition of blood and a selective supplement that contains colistin sulfate and oxolinic acid. However, it is also indispensable the incubation in the 5% CO2 atmosphere. Together with the previously mentioned difficulties, it needs the addition of blood, the use of antibiotics as a supplement. Moreover, it makes dificult the preparation of the medium and it doesn't guarantee a real inhibition of the undesirable accompanying microbiota due to the appearance, every time with more frequency, of resistance phenomenons to the antibiotics, mainly in the Gram negative organisms.

Furthermore, the necessity to incubate the medium in 5% CO₂ atmosphere makes annoying the isolation technique, when this method isn't directed to the identification or differentiation, so it is necessary to use culture media or biochemical and serological tests to identify Streptococcus target species.

In the before mentioned Manual, it is also described the composition of a Streptococcus/Staphylococcus selective medium that needs the addition of sterile blood and the antibiotic supplement, nalidixic acid and colistin sulfate. This formulation employment presents the same above-mentioned disadvantages, relative to the use of blood and antibiotics.

A selective medium for the rapid isolation of Streptococcus agalactiae and other streptococci related with the bovine mastitis: is Edwards (Modified) medium. It is described in the OXOID Manual 1995. It is a blood supplemented medium and it contains talium sulfate and crystal violet as inhibitors. This diagnostic allows the growth of Streptococcus agalactiae and other Streptococcus, using like identification principle only the esculin hydrolysis. The D group streptococci form a precipitate in the medium (black colonies) due to its metabolic activity and the other microorganisms give the negative answer (blue colonies) and cannot be differentiated.

The crystal violet used as a selective agent, can inhibit the growth of Streptococcus cell. The talium sulfate is also considered a highly toxic compound, noxious for the human health and extremely harmful for the environment. Subsequently it makes at the same time very annoying the production process, preparation and discarding of the medium wastes.

The Todd-Hewitt medium with gentamicin and nalidixic acid, Colistin Blood Agar with colistin and nalidixic acid, Mueller-Hinton Broth with 5% of serum and amikacin (Bosch, J.; Murillo, S.; Rico, M. y Salgado, M. 1998. Utilidad de un medio selectivo disco-caldo para la detección de estreptococo del grupo B en la vagina. Enferm. Infecc. Microbiol. Clin. 16(2)) and the Azide-Esculin-Oxolinic acid medium has been used already for the detection and identification of specific Streptococcus species. They have the similar to the previously mentioned disadvantages (Figueras, M. J; Inza, I.; Polo, F. and Guarro, J. 1998. Evaluation of the oxolinic acid-esculin-azide medium for the isolation and enumeration of faecal streptococci in a routine monitoring programme for bathing waters. Can. J. Microbiol. 44, p. 998-1002).

There have been requested or patented a group of inventions directed to the culture of specific Streptococcus species. Some of them are related below.

De la Rosa Fraile describes a dehydrated medium to identify group B Streptococcus in the EP 1 098 002 A1 patent presented in 2001.05.09 (Dehydrated immediate reconstruction culture medium to identify group B Streptococci (Streptococcus agalactiae) by detection of their pigment), where the differentiation is done by its own pigment production. The essence of the invention consists on adding Sephadex 200 as gelling agent to the Granada medium.

The limitations of the mentioned invention can be summarized undemearth:
It allows only the identification of Streptococcus agalactiae.
It is needed the addition of horse serum.
The prepared medium has a durability of less than 2 months due to the unstableness of the compounds.
There is necessity of supplemented addition of such antibiotics as metronidazole and colistin, that already presents the deficiencies similar to those described for the previous formulations.
The cultural characteristics of the microorganism cannot be observed, because the medium is semisolid.
   The preparation is complex, and it includes different steps, such as the preparation of the basal medium, the addition of the gel, the agitation to catch bubbles, the addition of the supplements and the incubation in Maria bath.

Years before the same author developed a formulation for the detection of Streptococcus agalactiae (De La Rosa Fraile, Manuel; Patente: ES 2088827, 1996.09.16. Culture medium for Streptococcus agalactiae which activates the formation of pigment by amylases and/or (G((alpha 1-4)G) (n(2) glucooligosaccharides added as chemical compounds, as polysaccharide hydrolysates or generated in the same medium using enzymes and polysaccharides) that contained antibiotics in supplement form, amylases, malto-oligosaccharides, starch for the production of the characteristic pigment of this microorganism. This formulation is complex to prepare, it contains labile enzymes added as supplement and complex chemical and labile compounds (malto-oligosaccharides). Therefore, its employment in the culture media production is not very common.

Another medium designed to the identification of group B Streptococcus hemolytic, is based on the production of a color orange pigment. It was developed by Tanaka Yoshihiro and Takahashi Hisayoshi (JP 9313171A2, 1997.12.09. Culture medium for group B hemolytic Streptococcus). The medium contains glucose, sodium piruvate, meat peptone, MgSO4, MOPS-phosphate, methotrexate and antibacterial agent (inactive for group B Streptococcus). By use of this formulation, other streptococci cannot be identified.

In the patent JP 59159797 (Kojima, H., et to the one. 1984.09.10. Culture medium for selective proliferation of Streptococcus hemolyticus), a selective medium for the propagation of Streptococcus hemolyticus is described. It contains lipids and fatty acids as specific selective agents to promote the growth of this specie. The employment of the soja bean in this medium makes complex its preparation, as well as the selective agents don't inhibit the growth of other target Streptococcus species.

Deyloff John protected a culture medium for the differentiation of Streptococcus mutans (U.S. Pat. No. 4,468,456, 1984.08.28. Medium for differentiating Streptococcus mutans) composed by phosphate salts, agar, yeast extract, sucrose and a pH indicator. The product preparation is complicated since it is composed of two solidified layers. To one of which it is necessary to add calcium phosphate suspension, which is difficult to disolve. The medium is not sufficiently inhibitory for other Gram-positive species.

Other media and methods have been developed for the culture of specific Streptococcus species to obtain certain metabolites, or even, for the diagnosis. This products in any case offers the possibility of differentiation or detection of a significant number of Streptococcus species. Among them can be mentioned Park et al. and Inoue et al. (U.S. Pat. No. 5496726, 1996. Streptococcus zooepidemicus medium and process for preparing hyaluronic acid; U.S. Pat. No. 4,306,024, 1981.12.15. Process for cultivation of hemolytic Streptococcus pyogenes).

Blareau Jean Pierre and collaborators (Patente: FR 2723960-A1, 1994.08.31. Cultures of Streptococcus thermophilus to activite beta-galactosidase elevee, leur proceeds d'obtention, et leurs utilisations) patented the culture medium for Streptococcus thermophilus that contains only milk protein hydrolysates, lactose and yeast extract. The medium isn't designed to the clinical diagnosis, neither to the control of the food quality and isn't selective medium for the Streptococcus detection.

Another group of inventions contrarily is directed to the culture of several genera or Streptococcus species, and don't allow a differentiation or appropriate identification among them, various examples make are cited next:

In the last years, it was increased the use of chromogenic and fluorogenic substrates that have been incorporated to the traditional media or in the new culture media, developed specifically for some microorganisms according to its metabolic demands. These chromogenic reactions possess certain advantages regarding to the conventional media, such as rapidity in obtaining of results, high specificity and sensibility due to the specific enzyme substrate degradation.

Manafi in various articles and extensive reviews mentioned the chromogenic and fluorogenic substrates more frequently employed as compounds of the culture media. In addition, he explains its possible usages in the identification of some species of Streptococcus. In the article “New developments in chromogenic and fluorogenic culture media” (Manafi, M. 2000. International Journal of Food Microbiology 60, p. 205-218), for example, he refers to some of these media, which present certain limitations or disadvantages:
Enterolert (IDEXX Laboratories Inc., Westbrook, Me.) and the Microtiter plate MUST (Sanofi, France) that contain specific fluorogenic substrates for the D-glucosidase, designed for the enterococci detection in water. Although it doesn't allow the diferenciación and identification of other streptococci and contain as a specific enzyme marker only fluorogenic substrate.
The agar was designed for the enterococci enumeration in sea water or fresh water. It contains nalidixic acid, cycloheximide, triphenyltetrazolium chloride and indoxyl βD glucoside as a substrate. The disadvantage of this medium is the use of antibiotics as selective agents, as well as possessing of a single enzymatic marker that disables the identification of other Streptococcus. There is necessity of long incubation periods (24-48 hours) and the sensibility of the medium is not too high.
The modification of this medium by reducing the triphenyltetrazolium chloride concentration and increasing the indoxyl βD glucoside concentration reduced the incubation period to 24 hours but it didn't increase the diagnostic possibilities of the medium.
Chromocult enterococci broth (CEB) (BBL) and the Readycult enterococci (MERCK) use the 5-bromo-4-chloro-3-indolyl-βD glucopyranoside as the substrate to identify Enterococcus and the sodium azide to inhibit other microorganisms. However, there appear false positive results in the medium, such as Leuconostoc, Lactococcus lactis and Aeromonas sp. Other streptococci are not identified and the medium contains the toxic ingredients.

The CHROMagar Orientation medium (BBL) allows the presumptive simultaneous identification of Gram positive, Gram negative organisms and yeasts (Carricajo, A.; Boiste, S.; Thore, J.; Aubert, G.; Gille, Y and Freydiere, A M. 1999. Comparative evaluation of five chromogenic media for detection, enumeration and identification of urinary tract pathogens. Eur J Clin Microbiol Infect Dis 18(11), p. 796-803; Samra, Z.; Heifetz, M.; Talmor, J.; Bain, E. and Bahar, J. 1998. Evaluation of use of a new chromogenic agar in detection of urinary tract pathogens. J Clin Microbiol., 36(4), p. 990-994; Hengstler, K A; Hammann, R. and Fahr, A M. 1998. Evaluation of BBL CHROMagar orientation medium for detection and presumptive identification of urinary tract pathogens. J Clin Microbiol. 36(5), p. 1464; Merlino, L; Siarakas, S.; Robertson, G J.; Funnell, G R.; Gottlieb, T and Bradbury, R. 1997. Evaluation of CHROMagar Orientation for differentiating and presumptive identification of gram-negative bacilli and Enterococcus species. J Clin Microbiol 35(8), p. 2190). Among the microorganisms that can be identified there are included the streptococci (groups D, B and C), although the groups B and C cannot be differentiated, so additional biochemical tests are required for their identification. The great diversity of colors makes extremely difficult the interpretation of the results and in occasions it is required to pay a special attention to the morphology, size and characteristics of the colonies (shine or opacity) that hinders even more the interpretation of the results. The similarity of colors of some streptococci with microorganisms of the genera Klebsiella, Enterobacter and Citrobacter, force to carry out additional tests that require more hours for carrying out. It is also prominent that the medium is sensitive to the light that changes the original color, also the incubation and reading should be made immediately.

The UTI medium (OXOID Inc.) designed for the presumptive identification and differentiation of urinary tract infection causing microorganisms. It contains the chromogenic substrate x-glu for the enterococci identification. This formulation presents as disadvantages that other streptococci cannot be identified, other Gram negative microorganisms grow that are presented generally in urinary infections in higher concentrations than Streptococcus and its use is restricted to urine samples.

In 1999, Alain Rambach patented a method for the identification of microorganisms with at least two chromogenic substrates (U.S. Pat. No. 5,962,251, 1999.10.05 “Method for the identification of microorganisms with at least two chromogens”). The medium contains agar, peptone, sodium chloride, yeast extract, meat extract and carbohydrates (glucose), as well as chromogenic indoxyl derived substrates (bromo- indoxyl, chloro-indoxyl, di-chloro-indoxyl, chloro-bromo-indoxyl, among other). Its main utility is the detection of presence or absence of yeasts belonging to genus Candida, especially Candida albicans and Candida tropicalis, although microorganisms of other genera, as well Gram-positive as Gram-negative grow with different color tonalities, and the streptococci among them. However, the diversity of developing organisms like in the previous case, delaies an appropriate interpretation of the results. There is a great competition for the susbtrates, so the streptococci can be inhibited and the differentiation among other species of Streptococcus (non-group D) becomes impossible.

Another group of chromogenic culture media developed for the detection, enumeration and identification of urinary pathogens that allows the identification specifically D group Streptococcus includes CPS ID2 (bioMerieux, France), Uriselect 3 (Sanofi Diagnostic) and Rainbow UTI (Biolog, USES) (Carricajo et to the., 1999. Comparative evaluation of five chromogenic media for detection, enumeration and identification of urinary tract pathogens. Eur J Clin Microbiol Infect Dis 18(11), p. 796-803; Navarro et al., 1996. Evaluation of chromogenic medium CPS ID2 (bioMerieux) in urine cultures. Enferm Infecc Microbiol Clin Apr; 14(4), p. 215-9; Reisner and Austin, 1997. Evaluation of CPS ID2 chromogenic agar as a single medium for urine culture. Diagn Microbiol Infect Dis Jul; 28(3), p. 113-7; Willinger and Manafi, 1995. Evaluation of a new chromogenic agar medium for the identification of urinary tract pathogens. Lett Appl Microbiol May 20(5), p. 300-2) and others (Bochner B. U.S. Pat. No. 5,464,755. 1995.10.07. Microbiological medium and method of assay; Yagupsky et al., 2000. Clinical evaluation of a novel chromogenic agar dipslide for diagnosis of urinary tract infections. Eur J Clin Microbiol Infect Dis 19, p. 694-698).

Chen, Chun-Ming and collaborators requested a patent of a method and medium for the detection of vancomycin-resistant enterococci (Chen, Chun-Ming; et to the one. Patent Application 20020132285-A1. 2002.09.19. Method and medium for detecting vancomycin-resistant enterococcus). The invention consists on the use of an ingredient that produces a reaction when being metabolized by the microorganisms, in this case the enzymes β-D-glucosidase and the pyrrolidonyl-arylamidase. It should be pointed out that the medium is unable to identify neither vancomycin sensitive enterococci nor other species of Streptococcus. Its preparation is troublesome, because it needs to be suplemented with antibiotics and the vancomycin doesn't inhibit some Gram-negative bacteria that can grow in the medium. Additional tests are required for the species identification.

In the application of U.S. Pat. No. 20020086278-A1. 2002.06.04, Gosnell and collaborators (Chromogenic half containing blood or hemin) describe a medium that contains blood or hemin and also chromogenic substrates such as: magenta, x-glucoside, x-glucuronide, Mag-phosphate, among others. The medium allows the growth and the identification of different species by morphological characteristic, the appearance of the diverse color tonalities, fluorescence and the hemolytic reactions. The invention can be considered as the nearest prototype and the medium presents the following disadvantages:
The presence of blood or hemin makes difficult the right interpretation of the chromogenic reactions. Moreover, the composition doesn't permit the inhibition of Gram-negative bacteria, among which there are a great diversity of microorganisms with enzymatic activity similar to the Streptococcus one.
The stability period of the medium is limited, because the presence of biological compounds (blood or hemin), and its preparation is annoying.
This culture medium not cotains inhibitory agents, isn't a selective medium, so it permits the development of other Gram-positive and Gram-negative genera that compete for the nutrients, and as the consequence the target microorganisms (Streptococcus) can't grow appropriately.
The use of supplemented blood and hemin makes complex the preparation of this medium and increases the risk of its contamination.
It is necessary the use of additional biochemical tests for the far along differentiation of Streptococcus, that needs an additional time to achieve an appropriate identification of the target germs. Specifically it is necessary to carry out an additional PYR test to differentiate Streptococcus pyogenes of the D group Streptococcus (Enterococcus).
For the identification of some microorganisms, it is necessary to keep in mind the morphology, the size and other characteristics of the colonies that are very variable because they depend on the sample type, initial concentration of the microorganism and accompanying microbiota, among other factors.
The possibility that microorganisms of other genera grow like Proteus whose fundamental characteristic is the spread formation in the medium, makes impossible the identification of the colonies.
Other microorganisms that have protein hydrolitic capacity can hydrolize the albumin and to interfere in the identification.
For the preparation of the medium, chromogenic substrates can be added to the fresh medium, prepared aseptically, since some of them are labiles to high temperatures, that makes complex their preparation.
The solutions of the chromogenic substrates, in some cases, form insoluble precipitate that may be more unstable after one-week storage that limits the period of usefulness of “in vitro” diagnostic.
When it is used like base for Chocolate Agar, the medium is incubated in atmosphere of 5% CO2.
Some chromogenic substrates need to be dissolved in the organic solvent dimethyl sulfoxide (DMSO) that it makes troublesome the preparation of the medium.
* In some cases (Example 3) the D group streptococci takes the same color of other microorganisms (blue), such: Candida albicans, Staphylococcus epidernidis and some Gram-negative organisms.
The substrates especially the magenta, may darken the color of the blood medium after the incubation that reduces the clarity of the lecture and makes difficult the observing of hemolytic reactions.
The chromogenic substrates that give coloration to the purple, red and magenta colonies doesn't hire appropriately with the red bottom of the medium.
   Popularization of the Invention

The objective of the present invention consists on providing a selective medium for the isolation and detection of species of Streptococcus genus, facilitating the differentiation of Streptococcus of clinical importance, such as: Streptococcus agalactiae, Streptococcus pyogenes and D group Streptococcus (Enterococcus), in early cultivation periods (2-24 hours) and with a high sensibility and analytic specificity.

The innovation of the proposed invention consists on a combination of nutritient bases specially selected in quantities from 15 to 58 g/l that guarantee a content of total nitrogen, fundamentally derived of the proteins, between 10 and 14%. Moreover it consists in a specially designed mixture of inhibitors of Gram-negative microorganisms in quantities from 0.55 to 1.63 g/l. Additionally it contains a mixture of chromogenic and/or fluorogenic substrates, which must reveal at least one specific enzyme of each species, in quantities from 0.3 to 1.5 g/l and indicative compounds of the deaminase activity, in quantities of 1 to 3 g/l.

The original mixture of nutrient bases is composed by extracts and hidrolysates of different origins, specifically selected among
Hydrolyzate of bovine blood of 5 to 10 g/l
Beef heart extract of lto 12 g/l
Enzymatic hidrolyzate of beef heart muscle of 1 to 5 g/l
Enzymatic hidrolyzate of milk proteins of 5 to 20 g/l
Enzymatic Hidrolyzate of Soja bean proteins of 2 to 15 g/l
Enzymatic Hidrolyzate of animal tisues of 1-5 g/l
Saccharomyces cerevisiae yeast autolyzate or hidrolyzate of 2 to 15 g/l

The never described peculiar mixture of inhibitors includes a combination of
Talium acetate from 0.55 to 1.6 g/l
Nalidixic acid from 0.005 to 0.030g/l

Another singular feature of the invention consists on the employment of the mixture of chromogenic and fluorogenic substrates that reveal the phosphatase, glucosidase and glucuronidase activity to differentiate the diverse Streptococcus species. It contains p-nitrophenylphosphate disodium salt (PNP), from 0.2 to 0.8 g/l; x-glu (5-bromo-4-chloro-3-indolyl-βD glucopyranoside, Mu-glu (4-methylumbelliferyl-β-D-glucopyranoside), Salmon-glu (6-chloro-3-indolyl-β-D-glucopyranoside) from 0.05 to 0.4 g/l and x-gluc (5-bromo-4-chloro-3-indolyl-β-D-glucuronide, cyclohexylammonium salt), Magenta-Gluc (5-bromo-6-chloro-3-indolyl-β-D-glucuronide, cyclohexylammonium salt) and MUG (4-methylumbelliferyl-β-D-glucuronide trihydrate) from 0.05 to 0.2 g/l.

These chromogenic substances combine in a very original form to guarantee the appearance of colors or fluorescences different for each species to detect. Among them can be mentioned
a) PNP+x-glu+Magenta-gluc or MUG
b) PNP+Salmon-glu+x-gluc or MUG
c) PNP+MU-glu+Magenta gluc or x-gluc

Another singular aspect of the invention, consists in that are added several compounds, which indicate the deaminase activity in the medium, such as trivalent metals salts, preferably ferric ammonium citrate in quantities from 0.5 to 1.0 g/l and aromatic amino acids, preferably phenylalanine and tryptophan, in quantities from 1.0 to 2.0 g/l.

To the before mentioned composition from 10.0 to 14.0 g/l of agar is incorporated. The obtained medium allows observing the cultural characteristics of the target organisms.

The formulated medium presents the pH values from 7 to 7.4 and its concentration in water may be varied from 25 to 80 g/l.

The medium permits, for the first time, isolate and detect exclusively different species of Streptococcus, fundamentally human pathogens, only by specific colony colors of certain genera and changes of the medium coloration.

For the first time is provided a selective medium that facilitates at the same time the differentiation of D group streptococci (Enterococcus) of the rest of them, and between the species of Streptococcus genus. For example, Streptococcus agalactiae and Streptococcus pyogenes that grows well while other Gram-positive and Gram-negative species are inhibited.

The advantages of the medium proposed in the following invention consist in that:
For the first time it is facilitated the differentiation between the pathogenic and most of non-pathogenic species of Streptococcus.
The medium is highly selective for Streptococcus, inhibits most of Gram negative bacterial species, except Proteus mirabilis ATCC 7002 which growth is very inhibited in the medium, moreover this organism can be identified by change of the medium color to one different to Streptococci species. Also very few species of other inhibited Gram positive organisms may develop a minimal growth in the medium and they take a coloration that doesn't interfere with the abundant growth of the target microorganisms.
The medium contains the complex and novel mixture of nutrient bases obtaind from dissimilar sources with various compositions, which provides the presence of amino acids, peptides, polypeptides, proteoses and peptones, and unexpectedly, they facilitate an abundant growth of the target species and allow the exclusion of labile substances such as hemin and blood.
The fact of special relevance and novelty is the inclusion of the blood enzymatic hydrolyzate in the before mentioned mixture. It guarantees the presence in the medium of blood derived substances or its fractions that are necessary for the Streptococcus growth and avoid the identification hindering. On the other hand, the digested blood proteins don't affect stability of the medium.
Due to the presence of enough cuantity of nutrients for the quick growth of streptococci in the medium, and the inhibitory substances that provides the absence of the other competitor organisms, the members of this genus can be developed faster and can be isolated and identified no later than after 6 to 24 hours of incubation.
The sensibility and specificity values are high for the reason that the streptococci cannot be confused with Gram negative bacteria, such as: Shigella and Escherichia coli, because these microorganism don't grow in the medium. Also a few Gram-positive species that are able to grow in the medium (Staphylococcus xylosus and Staphylococcus saprophyticus) have different cultural characteristics and are almost totally inhibited so cannot be confused with the rest of the microorganisms. If some strain presents the growth characteristics similar to Streptococcus, it can be differed by direct catalase test in the plate.
In the present invention are not used neither highly toxic substances nor environment pollutants. For this reason, the medium can be prepared easily such by industrial scale as in the laboratory, under appropriate and specific conditions of the activity.
The effectiveness of the inhibitors mentioned in the formulation avoids the use of labile antibiotic, so the medium preparation is simple and don't exist identification mistakes due to phenomenons of resistance to the antimicrobial agents.
The inhibitor combination and concentration in the medium and its ratio regarding to the nutrients makes possible that none Streptococcus species aren't inhibited in the medium. This fact makes the medium very suitable, as never before, for the specific diagnosis of this genus.
The faster diagnosis can be obtained because when the target streptococci are detected and differed appropriately, with high sensibility and specificity values, it is not necessary to carry out additional biochemical tests.
The preparation of this medium is extremaly simple because, in some cases, it can not be autoclaved, and in other, only one supplement must be added.
The stability of the prepared medium is large, because it contains inhibitors of most of environmental pollutants and doesn't contain labile substances (as the hemin, blood and antibiotics).
When the identification of the target bacteria is only carried out by the cultural characteristics (medium color and colonies), it excludes the necessity to observe detailedly morphological characteristics of the colonies, facilitating the interpretation of the results for a trained but not specialized personnel.
The use of more than one enzymatic marker for the differentiation of the diverse species of the Streptococcus genus increases the diagnostic specificity and sensibility.
The formulation reported in the application facilitates to prepare the medium to different concentrations for multiple presentations (for example, to simple concentration in gelled medium, to double concentration in the membrane filtration techniques, or in broth form, and even to a higher concentration for miniaturized systems).
With this medium, it is possible to substitute the employment of different traditional or chromogenic media used in the identification of different species within Streptococcus genus.
The proposed formulation facilitates its employment in the screening of Streptococcus in clinical or veterinary samples, in waters and other elements of the environment as well as in foods.
It is achieved the isolation and simultaneous identification in a single step, with the consequent reduction of time, of resources and personal.

### DETAILED DESCRIPTION OF THE INVENTION

For the definition of the composition of the medium, the components used in the preparation were preferably solids. The description of the steps that must be done for the preparation of the medium is presented below.

The mixture of nutrient bases which total nitrogen content is between 10 and 14%, is sifted to achieve uniformity in the size of the particles.

The quantity of these ingredients of proteic origin is varied from 15 to 58 g/l.

Specifically the cuantity ranges are described below.
Bovine blood hidrolyzate from 5 to 10 g/l,
Beef heart extract from I to 12 g/l,
Beef heart muscle enzymatic hidrolyzate from 1 to 5 g/l,
Milk protein enzymatic hidrolyzate from 5 to 20 g/l,
Soja bean protein enzymatic hidrolyzate from 2 to 15 g/l,
Animal tissue enzymatic hidrolyzate from 1 to 5 g/l,
Saccharomyces cerevisiae yeast autolyzate or hidrolyzate from 2 to 15 g/l.

The selective agents are selected among: talium acetate from 0.55 to 1.6 g/l and nalidixic acid from 0.05 to 0.030 g/l. By the same way they are sifted to stay ready for mixing with rest of the compounds.

The chromogenic and fluorogenic compound to detect the glucosidase, phosphatase and glucuronidase activity in quantities from 0.3 to 1.5 g/l, are premixtured. Furthermore, they may be milled and sifted previously to the homogenization. If the mixing procedure is efficient weighing the premixture cuantity equal to the sum of the quantities of each component in the formulation, an appropriate distribution of each one must be achieved.

According to the medium purpose, chromogenic and fluorogenic substrates may be selected among
p-nitrophenylphosphate disodium salt (0.2 to 0.8 g/l);
x-glu (5-bromo-4-chloro-3-indolyl-βD-glucopyranoside), Mu-glu (4-methylumbelliferyl-βD-glucopyranoside), Salmon-glu (6-chloro-3-indolyl-βD-glucopyranoside) (0.05 to 0.4 g/l);
x-gluc (5-bromo-4-chloro-3-indolyl-βD-glucuronide cyclohexylammonium salt), Magenta-gluc (5-bromo-6-chloro-3-indolyl-βD-glucuronide ciclohexilamonio salt) and MUG (4-methylumbelliferyl-βD-glucuronide trihydrate)(0.05 to 0.2 g/l).

There was carried out the conformation of the mixture of chromogenic and fluorogenic substances that may be degradable by at least one enzyme of each specie. The combinations that guarantee the appearance of colors or different fluorescence for each specie are described next:
a) PNP+x-glu+Magenta-gluc or MUG
b) PNP+Salmon-glu+x-gluc or MUG
c) PNP+Mu-glu+Magenta-gluc or x-gluc

The gelling agent, preferably agar, with hardness between 400 and 700 g/cm², is used in a range of 10.0 to 14.0 g/l, specifically for the solid medium. Before be added to the medium it must be dried and sifted.

All the before mentioned compounds, as well as the premixture, are united and homogenized until achieving an uniform mix with a pH value between 7.0 and 7.4. The mixture is distributed in tightly closed flasks, which are protected from the light and maintained at environmental temperature.

The powder is suspended in destilled or deionized water in quantities from 30 to 80 g/l.

The composition can be made at laboratory scale, weighing the ingredients separately inside a recipient and maintaining the before mentioned proportions. Later little by little is added the water on the powder mixture, until achieving complete dissolution. Mix thoroughly the suspension and allow to rest at least 10 minutes. If solid medium must be prepared, this may be either sterilized or not foresaw to the addition of the p-nitrophenylphosphate disodium salt. If cautions have been taken in the preparation, it is enough the heating until to boil, because the medium is highly inhibitory for environmental microorganisms that could spoil it. Otherwise, the medium must be sterilized for 10 minutes at 115° C. and then, after it is cooled to 45-50° C., sterile filtered PNP solution is added. In case of the liquid medium, it is always sterilized in autoclave and then the sterile supplement is added. When the broth is prepared, it must be distributed in the final containers.

In dependence of the medium consistency, solid or liquid, different inoculation methods may be used.

Once inoculated, they are incubated at 30 to 37° C. for at least from 2 to 6 hours in case of the liquid medium and from 18 to 24 hours in case of the solid medium.

The reading of the results is carried out in the case of the solid medium, observing the color of the colonies isolated in the surface of the medium. For the liquid medium only is observed the change of medium coloration and the fluorescence. So the D group streptococci can be differed from the rest of the species of this genus. The staphylococci, generally, are not developed in the medium or may be very inhibited and observed as colorless small colonies (Staphylococcus saprophyticus) or with the colony colors different from the streptococci and so don't interfere in the identification of the target organisms.

The enterococci is observed of rose or blue color or with fluorescens according to the combination of the used substrates, while in dependence of the used combination the streptococci is observed yellow, yellow greenish, green, magenta, blue or fluorescent, whenever is used a color substrate different to the employed to reveal the enterococci presence.

### EXAMPLES OF REALIZATION

### Example No. 1

In this experiment, the effects of a series of nutrient base mixtures were studied as nutritional components in the formulation. In the experiments 3 variants were designed whose composition 1 appear in the table. The ingredients were reconstituted in 100 mL of water and proceeded to prepare them according to the detailed description of the present invention.

The evaluation of the promotion capacity of growth was carried out with collection strains in comparison with reference medium (Selective Broth of Streptococci Enrichment, Merck, lot: 86444327).

In each tube was inoculated 0.1 mL of standardized solution at 50% of transmitance of each microorganism.

The curves of growth appear in the FIGS. 1, 2 and 3.

The results of the promotion of streptocococi growth were highly satisfactory. In the case of Streptococcus agalactiae it was observed that the 3 characteristic compositions of the present invention promoted with more intensity the cell development during the first 8 hours with regard to the control medium and even the variant 1 promoted the growth in a very superior form until the 9 hours.

In the case of Streptococcus faecalis a bigger acceleration of the growth was observed in the first 4 hours, in the experimental variants, with regard to the control, and in the case of Streptococcus pyogenes, starting from the 6 hours the growth was significantly superior in the new proposed compositions.

TABLA 1
Composition of nutrient bases of the different experimental variants

| | Composition | | |
|---|---|---|---|
| | (g/100 mL) | | |
| Component | V1 | V2 | V3 |
| Saccharomyces cerevisiae autolyzate or yeast | 0.32 | 0.32 | 0.32 |
| hydrolyzate | | | |
| Beef heart extract | 0.24 | 0.24 | 0.24 |
| Milk protein enzymatic hydrolyzate | 0.97 | 0.97 | 0.97 |
| Soja bean protein enzymatic hydrolyzate | 0.32 | — | — |
| Beef heart muscle enzymatic hydrolyzate | — | — | 0.22 |
| Animal tissue enzymatic hydrolyzate | — | 0.46 | — |
| Total of the mixture | 1.85 | 1.99 | 1.75 |
| Total Nitrogen of the mixture | 13.30 | 12.36 | 12.31 |

### Example No. 2

The composition was prepared with the ingredients according to example 1, but weighed for separate inside a Erlenmeyer flask. As agents promoters of the growth and enzymatic markers were used the following ingredients:

| Component | g/100 mL |
|---|---|
| Milk protein enzymatic hydrolyzate | 0.97 |
| Saccharomyces cerevisiae autolyzate or yeast hydrolyzate | 0.32 |
| Beef heart extract | 0.24 |
| Soja bean protein enzymatic hydrolyzate | 0.32 |
| p-nitrophenylphosphate disodium salt | 0.04 |
| x-glu (5-bromo-4-chloro-3-indolyl-βD-glucopyranoside) | 0.02 |

In the formulation agar was not used. It was added to the mixture of ingredients solid 100 mL of water desionizada and proceeded to the preparation of the composition as it is exposed in the detailed description.

Collection strains of Streptococcus pyogenes ATCC 19615 were inoculated, Streptococcus agalactiae ATCC 12386, Enterococcus faecalis ATCC 29212, Enterococcus faecalis ATCC 19433, Enterococcus faecium ATCC 6056. This strains were inoculated in a direct form with a standardized solution at 50% of transmitance in the tubes of each one.

It was achieved at the 18 hours that the medium appear blue-greenish in the case of Enterococcus faecalis ATCC 29212 and 19433. The strain of Enterococcus faecium showed dark blue coloration, Streptococcus agalactiae appear green and Streptococcus pyogenes were observed of yellow-greenish color.

The results show the speed of the microorganism answer in the time and the possibility of differentiating the enterococci (group D) of the rest of estreptococci because they exhibits a coloration among green-blue to blue and the streptococi, such as: Streptococcus pyogenes and Streptococcus agalactiae to show a coloration greenish yellow.

To check the certainty of the selection of chromogenic substrate x-glu was prepared a medium whose similar composition to the one described previously, with the difference that the esculin was used. The used quantity was of 0.1 g/100 mL, iron salts were also added in a concentration of 0.05 g/100 mL.

These ingredients were weighed had separated in a Erlenmeyer flask, later on 100 mL of deionized water was added and proceeded to the preparation.

Collection strains of Streptococcus pyogenes ATCC 19615 were inoculated, Streptococcus agalactiae ATCC 12386, Enterococcus faecalis ATCC 29212 and some isolated strains of clinical samples (4 enterococci and 4 hemolytic streptococci). This strains were inoculated in a direct form with a standardized solution at 50% of transmitance in the tubes of each one.

The obtained answer for both strain, Enterococcus faecium and Enterococcus faecalis was a change of medium color to black, what masks the other enzymatic reactions to which some species can be positive.

Streptococus pyogenes and Streptococcus agalactiae, both strains appear of color green olive tree. The 4 isolated strains of enterococi gave black color, the same aswer exhibit by 3 hemolytic streptococci. Only a hemolytic microorganism was observed of green olive tree color the same as Streptococus pyogenes and Streptococcus agalactiae.

The obtained results were appreciated in an early form, however the blackish coloration as a result of esculin hydrolysate whereby metabolic activity of Group D enterococci, limits the identification, because it disables the reading of the rest of the reactions.

As conclusion the experiment allowed to check that accepted of the inclusion of the x-glu as ingredient in the formulation of the medium, and not the esculin use.

### Example No. 3

The composition of the medium was prepared according to the example 2, with the difference that was increased the concentration of the nutritious components and the enzymatic markers. They were weighed for separate inside a Erlenmeyer flask following quantities:

| COMPONENT | g/100 mL |
|---|---|
| Milk protein enzymatic hydrolyzate | 1.94 |
| Saccharomyces cerevisiae autolyzate or yeast hydrolyzate | 0.64 |
| Beef heart extract | 0.48 |
| Soja bean protein enzymatic hydrolyzate | 0.64 |
| p-nitrophenylphosphate disodium salt | 0.08 |
| x-glu (5-bromo-4-chloro-3-indolyl-βD-glucopyranoside) | 0.04 |

It was added to the mixture of solid ingredients 100 mL of deionized water and proceeded to the preparation.

Collection strains were inoculated, such as: Streptococcus pyogenes ATCC 19615, Streptococcus agalactiae ATCC 12386, Enterococcus faecalis ATCC 29212, Enterococcus faecalis ATCC 19433, Enterococcus faecium ATCC 6056 and some isolated strains: Enterococcus avium (isolated). This strains were inoculated in a direct form in the wells of a polypropylene module (Module 250 pp, NUNC, lot: 046247) adding 0.1 mL of a standardized solution at 3.0 McFarland in 0.1 mL of medium.

At the 2 hours it was observed changes of medium coloration to blue-greenish for Enterococcus faecalis ATCC 29212, blue for Enterococcus faecalis ATCC 19433, Enterococcus avium appears yellow-greenish, Enterococcus faecium gave clear blue color, Streptococcus pyogenes and Streptococcus agalactiae were shown of intense yellow color.

The answer stayed in the time (until the 6 hours). The results demonstrated the speed in the answer to the enzymatic reactions, and the possibility of developing a rapid test for the identification of species of Streptococcus genus.

### Example No. 4

The composition of the ingredients was prepared according to the example 2, with the addition of an gelling agent (agar) in a concentration of 6.5 g/500 mL. The components were weighed had separated in a Erlenmeyer flask and it was added to the mixture of solid ingredients 500 mL of deionized water. Later on proceeded to the preparation according to the example 1.

Collection strains were inoculated, such as: Streptococcus pyogenes ATCC 19615, Enterococcus faecalis ATCC 29212, Enterococcus faecalis ATCC 19433, Enterococcus faecium ATCC 6056.

They were inoculated by spreading the microorganism onto the surface of the medium, until achieving isolated colonies.

It was achieved at the 24 hours a green-clear coloration of the colonies with change of the medium to yellow of Streptococcus pyogenes. The strains of Enterococcus faecalis ATCC 29212 and 19433 showed characteristic cultural similar, appear a blue coloration with a small blue halo around. As for Enterococcus faecium, the colonies were observed smaller than the rest of the enterococi, of blue color with a small blue halo around.

### Example No. 5

The composition of the medium was prepared weighing the ingredients had separated in a Erlenmeyer flask according to the example 4, with the difference that the enzymatic markers are in a smaller concentration (0.2 g/l p-nitrophenylphosphate disodium salt and 0.1 g/l of 5-bromo-4-chloro-3-indolyl-βD-glucopyranoside).

The preparation method was the one described in the example 1.

They were inoculated by spreading the microorganism onto the surface of the medium, until achieving isolated colonies, collection strains of: Streptococcus pyogenes ATCC 19615, Enterococcus faecalis ATCC 29212, Enterococcus faecium ATCC 6056, Staphylococcus aureus ATCC 25923 and an isolated strain of Enterococcus avium.

The reading carried out at the 24 hours of the evaluation of the composition with the 20 collection strains was observed as it is shown in the table No. 2.

TABLE NO. 2
Characteristics of growth and of the microorganism colonies in the
solid medium.

| | Characteristic of the colonies |
|---|---|
| Strain | and of the medium |
| Streptococcus pyogenes ATCC 19615 | Greenish colonies with change |
| | of medium color to yellow |
| Enterococcus faecalis ATCC 29212 | Blue colonies |
| Enterococcus faecium ATCC 6056 | Blue colonies |
| Staphylococcus aureus ATCC 25923 | White colonies with change of |
| | medium color to yellow |
| Enterococcus avium (isolated) | Blue colonies |

### Example No. 6

The composition was prepared with the ingredients according to the example 2, with the difference that the chromogenic substrates x-glu (5-bromo-4-chloro-3-indolyl-βD-glucopyranoside) was substituted for Mu-glu (4-methylumbelliferyl-βD-glucopyranoside) in a concentration of 0.05 g/l. The preparation method went similar to the example 2.

In the evaluation collection strains were used of: Enterococcus faecalis ATCC 29212, Enterococcus faecium ATCC 6056, Streptococcus lactis ATCC 11454 and an isolated strain of Enterococcus avium. This strains were inoculated in a direct method in the wells of a polypropylene module (Module 250 pp, NUNC, lot: 046247) adding 0.1 mL of a standardized solution at 2.0 McFarland in 0.1 mL of medium.

It was achieved starting from the 2 hours to detect the growth of the rehearsed microorganisms and the fluorescence of color blue under ultraviolet light (365 nm) of Enterococcus faecalis and Enterococcus faecium.

The reading at the 6 hours showed that all the inoculated strains possessed glucosidase activity, because they responded with a blue fluorescence color when being lit with ultraviolet light.

It was demonstrated that it is possible the use of the fluorogenic substrate Mu-glu (4-methylumbelliferyl-βD-glucopiranoside), because the detection of streptococci (Enterococcus) was rapid.

### Example No. 7

The medium composition was prepared weighing the ingredients had separated in a Erlenmeyer flask. Two inhibitors mixtures were rehearsed in the solid medium. Next it is described the composition of growth promoter agents, enzymatic markers and used inhibitors:

| | g/200 mL | |
|---|---|---|
| COMPONENT | V1 | V2 |
| Saccharomyces cerevisiae autolyzate or yeast hydrolyzate | 1.944 | 1.944 |
| Beef heart extract | 0.648 | 0.648 |
| Milk protein enzymatic hydrolyzate | 0.486 | 0.486 |
| Soja bean protein enzymatic hydrolyzate | 0.648 | 0.648 |
| p-nitrophenylphosphate disodium salt | 0.04 | 0.04 |
| Salmon-glu (6-chloro-3-indolyl-βD-glucopyranoside) | 0.02 | 0.02 |
| x-gluc (5-bromo-4-chloro-3-indolyl-βD-glucuronide | 0.02 | 0.02 |
| cyclohexylammonium salt) | | |
| Talium acetate | 0.12 | 0.12 |
| Nalidixic acid | 0.003 | 0.002 |
| Agar | 2.6 | 2.6 |

Next proceeded to the preparation according to the example 1.

The used inoculation method was spreading the microorganism onto the surface of the medium, until achieving isolated colonies. In the evaluation collection strains were used of: Proteus mirabilis ATCC 7002, Proteus mirabilis ATCC 12433, Staphylococcus epidermidis ATCC 12228, Staphylococcus aureus ATCC 25923, Staphylococcus xylosus ATCC 29971, Staphylococcus saprophyticus ATCC 15305, Enterococcus faecium ATCC 6056, Streptococcus agalactiae ATCC 12386, Streptococcus faecalis ATCC 29212 and the isolated strain of Enterococcus avium.

It was carried out the reading at the 24 hours, the results of the behavior of the microorganisms in the medium are shown in the table No. 3. It is necesary highlight that the results obtained in both variants (V1 and V2) they were similar.

TABLE NO. 3
Characteristic of the medium and colonies

| Strain | Growth | Color of the medium and colonies |
|---|---|---|
| Streptococcus agalactiae ATCC 12386 | + | Green-blue colonies with change of |
| | | medium coloration to yellow |
| Enterococcus faecalis ATCC 29212 | + | Rosy colonies |
| Enterococcus faecium ATCC 6056 | + | Rosy colonies |
| Enterococcus avium (isolated) | + | Rosy colonies |
| Staphylococus saprophyticus | poor | White colonies |
| ATCC 15305 | | |
| Staphylococcus xylosus ATCC 29971 | + | Blue colonies |
| Staphylococcus aureus ATCC 25923 | + | White colonies |
| Proteus mirabilis ATCC 7002 | − | — |
| Proteus mirabilis ATCC 12433 | − | — |

Legend
+: good growth
−: inhibited growth

The strains of Proteus mirabilis stayed inhibited, evidencing the inhibitory power of the medium for the Gram negative organisms.

Some strains of Staphylococcus were developed in the medium, however they took colorations different to the streptococci, but it was also carried out the rapid test of catalase, adding a drop of the reagent in the medium, being achieved the complete differentiation of both genera.

### Example No. 8

The composition of the ingredients was prepared according to example 7, the components were weighed had separated in a Erlenmeyer flask with the difference that x-gluc was substituted (5-bromo-4-chloro-3-indolyl-βD-glucuronide cyclohexylammonium salt) for Magenta-gluc (5-bromo-6-chloro-3-indolyl-βD-glucuronide cyclohexylammonium salt).

The preparation method went similar to the example 7.

Collection strains of Proteus mirabilis ATCC 7002 were inoculated, Staphylococcus epidermidis ATCC 12228, Staphylococcus aureus ATCC 25923, Staphylococcus xylosus ATCC 29971, Enterococcus faecium ATCC 6056, Streptococcus agalactiae ATCC 12386, Streptococcus pyogenes ATCC 19615, Enterococcus faecalis ATCC 29212, Staphylococcus saprophyticus ATCC 15305 and the isolated strain of Enterococcus avium.

The results of the behavior of the microorganisms in the medium are shown in the Table No. 4.

TABLE NO. 4
Characteristic of the medium and colonies

| | | Color of the medium and |
|---|---|---|
| Strain | Growth | colonies |
| Streptococcus pyogenes ATCC 19615 | + | White colonies with change of |
| | | medium coloration to yellow |
| Streptococcus agalactiae ATCC 12386 | + | Magenta colonies with slight |
| | | change of medium coloration to |
| | | yellow |
| Enterococcus faecalis ATCC 29212 | + | Rosy colonies |
| Enterococcus faecium ATCC 6056 | + | Rosy colonies |
| Enterococcus avium (isolated) | + | Rosy colonies |
| Staphylococus saprophyticus ATCC 15305 | − | — |
| Staphylococcus xylosus ATCC 29971 | + | Magenta colonies |
| Staphylococcus aureus ATCC 25923 | + | White colonies |
| Proteus mirabilis ATCC 7002 | − | — |

Legend
+: good growth
−: inhibited growth

An early and differentiated growth of the species of Streptococcus was observed, for the different colorations developed in the medium.

Some strains of Staphylococcus were developed in the medium and they took colorations different to streptococci, although Staphylococcus xylosus appears of a similar color to Streptococcus agalactiae, but it is posible diffentiate because he don't change the medium color to yellow. Nevertheless was carried out rapid test of catalase, adding a drop of the reagent in the medium, and was achieved the identification of Staphylococcus genus, product of the liberation of H₂ and O₂ (formation of bubbles).

### Example No. 9

The composition was prepared according to the example 8, with the difference that X-gluc was substituted by the fluorogenic substrate MUG, in a concentration of 0.05 g/l. It was added to the mixture of solid ingredients deionized water and proceeded to the preparation of the composition until their gelling process as it is described in the example number 1.

They were inoculated by spreading the microorganism onto the surface of the medium collection strains of Proteus mirabilis ATCC 7002, Staphylococcus aureus ATCC 25923, Staphylococcus xylosus ATCC 29971, Enterococcus faecium ATCC 6056, Streptococcus agalactiae ATCC 12386, Streptococcus pyogenes ATCC 19615, Streptococcus faecalis ATCC 29212, Staphylococcus saprophyticus ATCC 15305 and the isolated strain of: Enterococcus avium. The reading was carried out at the 24 hours, according to the table No. 5

TABLE NO. 5
Characteristics of the growth and the colonies at the 24 hours of
incubation

| | | | Fluo- |
|---|---|---|---|
| | | Characteristic of the | res- |
| Strain | Growth | colonies and medium | cence |
| Streptococcus agalactiae | + | White colonies with | + |
| ATCC 12386 | | change of medium color | |
| | | to yellow | |
| Streptococcus pyogenes | + | White colonies with | − |
| ATCC 19615 | | change of medium color | |
| | | to yellow | |
| Enterococcus faecalis ATCC | + | Rosy colonies with | − |
| 29212 | | change of medium color | |
| | | to yellow | |
| Enterococcus faecium ATCC | + | Rosy colonies | − |
| 6056 | | | |
| Enterococcus avium (aislado) | + | Rosy colonies | − |
| Staphylococcus xylosus | ± | White colonies | − |
| ATCC 29971 | | | |
| Staphylococcus aureus ATCC | ± | White colonies | − |
| 25923 | | | |
| Staphylococcus saprophyticus | − | — | − |
| ATCC 15305 | | | |
| Proteus mirabilis ATCC 7002 | − | — | − |

Legend
+: good growth and positive fluorescence
(: scarce growth
−: inhibited growth

### Example No. 10

The medium composition was prepared weighing the ingredients had separated in a Erlenmeyer flask. The medium was prepared with the same relationship of nutrient bases that the example 7, to exception that bovine blood enzymatic hidrolyzate was added in quantities of 1.911 g/300 mL. Two different enzymatic markers were also rehearsed in concentration of 1.0 g/l with the addition of 0.5 g/l ferric ammonium citrate and two concentrations different from nalidixic acid 0.015 g/l and 0.010 g/l. Next it is described the composition of enzymatic markers and used inhibitors:

| | g/300 mL | g/300 mL |
|---|---|---|
| COMPONENT | V1 | V2 |
| P-Nitrophenylphosphate | 0.06 | 0.06 |
| disodium salt | | |
| Salmon-glu (6-chloro-3-indolyl-βD- | 0.0225 | 0.0225 |
| glucopyranoside) | | |
| x-gluc (5-bromo-4-chloro-3-indolyl- | 0.0225 | 0.0225 |
| βD-glucuronide | | |
| cyclohexylammonium salt) | | |
| Talium acetate | 0.18 | 0.18 |
| Nalidixic acid | 0.0045 | 0.003 |
| Tryptophan | 0.3 | — |
| Phenylalanine | — | 0.3 |
| Ferric ammonium citrate | 0.15 | 0.15 |
| Agar | 3.9 | 3.9 |

Proceeded to the preparation according to the example 1.

The used inoculation method was: spreading the microorganism onto the surface of the medium and in the case of the organisms used as positive controls it was also inoculated by dilutions (10⁻⁴ and 10⁻⁵). In the evaluation collection strains were used of: Proteus mirabilis ATCC 7002, Proteus mirabilis ATCC 12433, Citrobacter freundii ATCC 8090, Citrobacter freundii ATCC 10625, Enterococcus faecium ATCC 6056, Streptococcus agalactiae ATCC 12386, Enterococcus faecalis ATCC 29212 and the isolated strain of Enterococcus avium.

The results of the behavior of the microorganisms in the medium are shown in the table No. 6.

TABLE NO. 6
Evaluation of the behavior of the different microorganisms
(incubation 35° C. for 24 h).

| | | | Promotion of |
|---|---|---|---|
| | | Characteristic and color | the growth |
| Strain | Media | of the isolated colonies | UFC/mL |
| Streptococcus agalactiae | Experim. | Green-blue colonies with | 365 |
| ATCC 12386 | | medium yellow | |
| | ATS | White colonies | 330 |
| Enterococcus faecalis ATCC | Experim. | Rosy colonies | 600 |
| 29212 | ATS | White colonies | 445 |
| Enterococcus faecium ATCC | Experim. | Rosy colonies | 310 |
| 6056 | ATS | White colonies | 65 |
| Enterococcus avium (isolated) | Experim | Rosy colonies | 240 |
| | ATS | White colonies | 110 |
| Citrobacter freundii ATCC | Experim. | Inhibited | — |
| 8090 | ATS | White colonies | |
| Citrobacter freundii ATCC | Experim. | Inhibited | — |
| 10625 | ATS | White colonies | |
| Proteus mirabilis ATCC 7002 | Experim. | Inhibited | — |
| | ATS | White colonies | |
| Proteus mirabilis ATCC 12433 | Experim. | Inhibited | — |
| | ATS | White colonies | |

Experim: Composition of the present invention
ATS: AGAR TRIPTONE SOJA
UFC/mL: Units Forming Colony per each mL of dilution 10⁻⁵

In the medium it is observed the growth of Streptocococcus agalactiae, Enterococcus faecalis, Enterocccus faecium and Enterococcus avium to high dilutions, the differentiated growth of the different species is demonstrated inside the Streptococcus genus.

Strains of Gram negative microorganisms was inoculated, using as reference the traditional medium Agar Triptone Soja. When carrying out the comparison with the control medium was proven the inhibitory power of the formulation of this invention when observing the total inhibition of the Citrobacter and Proteus genera.

### BRIEF DESCRIPTION OF THE FIGURES

FIG. 1: Growth curve of Enterococcus faecalis ATCC 29212 in the different nutrient base mixtures.

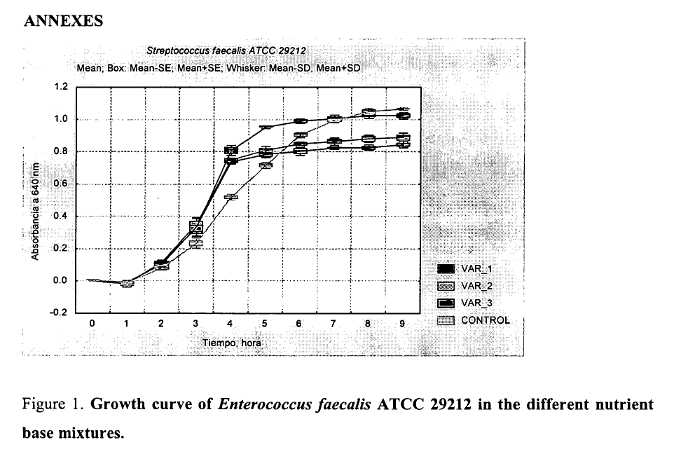

FIG. 2: Growth curve of Streptococcus agalactiae ATCC 12386 in the different nutrient base mixtures.

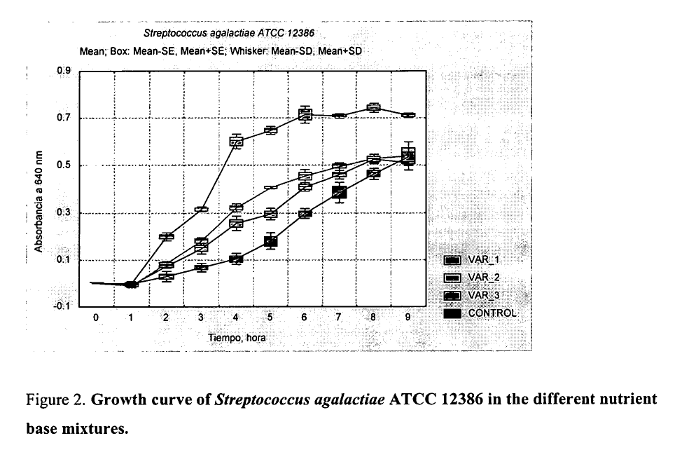

FIG. 3: Growth curve of Streptococcus pyogenes ATCC 19615 in the different nutrient base mixtures.

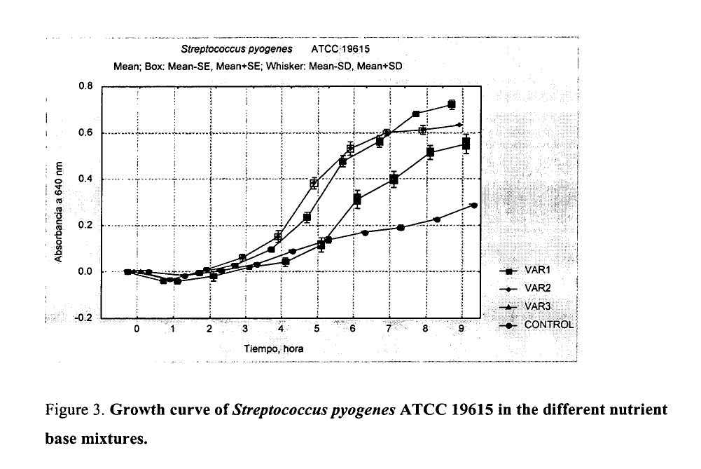

1-16. (canceled)

17. Selective culture medium for the isolation and early detection of species of the Streptococcus genus, characterized to contain a nutrient group of nutrient bases in quantities from 15 to 58 g/ that guarantee a total nitrogen content, derived fundamentally of the proteins from 10 to 14%, selected among the Bovine blood hidrolyzate, Beef heart extract, Beef heart muscle enzymatic hidrolyzate, Milk protein enzymatic hidrolyzate, Soja bean protein enzymatic hidrolyzate, Animal tissue enzymatic hidrolyzate, Saccharomyces cerevisiae yeast autolyzed or hidrolyzate, and their mixtures; a inhibitor group in quantities of 0.55 to 1.6 compound g/l for talium acetate and nalidixic acid; a degradable chromogenic and fluorogenic substance mixture, for at least an enzyme of each species in quantities of 0.3 to 1.5 g/l, selected among the p-nitrophenylphosphate disodium salt, 5-bromo-4-chloro-3-indolyl-βD-glucopyranoside, 4-methylumbelliferyl-βD-glucopiranoside, 6-chloro-3-indolyl-βD-glucopyranoside, 5-bromo-4-chloro-3-indolyl-βDglucuronide cyclohexylammonium salt, 5-bromo-6-chloro-3-indolyl-βD-glucuronide ciclohexylammonium salt and 4-methylumbelliferyl-βD-glucuronide trihydrate and indicative compounds of deaminase activity, in quantities of 1 to 3 g/l.

18. Culture medium, according to claim 17, characterized to contain nutrient bases specified in the following quantities within the medium:
Bovine blood hidrolyzate of 5-10 g/l
Beef heart extract of 1-12 g/l
Beef heart muscle enzymatic hidrolyzate of 1-5 g/l
Milk protein enzymatic hidrolyzate of 5-20 g/l
Soja bean protein enzymatic hidrolyzate of 2-15 g/l
Animal tissue enzymatic hidrolyzate of 1-5 g/l
Saccharomyces cerevisiae yeast autolizate or hidrolyzate 2-15 g/l

19. Culture medium, according to claim 17, characterized to contain Gram negative growth inhibitors, in the following quantities within the medium:
Talium acetate of 0.55 to 1.6 g/l
Nalidixic acid of 0.005 to 0.030 g/l

20. Culture medium, according to claim 17, characterized to contain degradable chromogenic and fluorogenic substances for at least one from the organisms to identify, selected in the following quantities within the medium:
p-nitrophenylphosphate disodium salt (PNP) of 0.2 to 0.8 g/l
5-bromo-4-chloro-3-indolyl-βDglucopyranoside (x-glu), 4-Methylumbelliferyl-βD-glucopyranoside (Mu-glu), 6-chloro-3-indolyl-βD-glucopiranoside (Salmon-glu) of 0.05 to 0.4 g/l
5-bromo-4-chloro-3-indolyl-βD-glucuronide cyclohexylammonium salt (x-gluc), 5-bromo-6-chloro-3-indolyl-βD-glucuronide cyclohexylammonium salt (Magenta-Gluc) and 4-Methylumbelliferyl-βD-glucuronide trihydrate (MUG) of 0.05 to 0.2 g/l.

21. Culture medium, according to claim 17, characterized to contain deaminase activity indicative compounds in the following quantities within the medium:
trivalent metal salts, preferably ferric ammonium citrate in quantities of 0.5 to 1.0 g/l.
aromatic amino acids, preferably tryptophan and phenylalanine in quantities of 1 to 2 g/l.

22. Culture medium, according to claim 17, characterized by conformation of the mixture of degradable chromogenic and fluorogenic substances for at least an enzyme of each species, in such combinations that guarantee the appearance of colors or different fluorescence for each species, like it continues next:
a) PNP+x-glu+Magenta-gluc or MUG
b) PNP+Salmon-glu+x-gluc or MUG
c) PNP+Mu-glu+Magenta-gluc or x-gluc

23. Culture medium, according to claim 17, characterized to also contain agar with hardness between 400 and 700 g/cm2 in quantities of 10 and 14 g/l for the preparation in the solid form.

24. Culture medium, according to the recovery 17, characterized to a pH value from 7 to 7.4.

25. Culture medium, according to claim 17, characterized because the same one gets ready adding quantities from 25 to 80 g/l of distilled or deionized water.
